(19) United States
(12) Patent Application Publication
(10) Pub. No.: US 2004/0133090 A1
(43) Pub. Date: Jul. 8, 2004

(54) Sanitary article for an organism state analysis and a method of an organism state analysis

(76) Inventors: Ilya Andreevich Dostoinov, Moscow (RU); Dmitry Yurievich Bykhovsky, Moscow (RU); Viktor Andreevich Pastukhov, Moscow (RU); Valery Lvovich Kuznetsov, Moscow (RU)

Correspondence Address: WILLIAM COLLARD, COLLARD & ROE, P.C., 1077 NORTHERN BOULEVARD, ROSLYN, NY, 11576, US

(21) Appl. No.: 10/476,396

(22) PCT Filed: Apr. 18, 2002
(86) PCT No.: PCT/RU02/00178

(30) Foreign Application Priority Data
Jul. 12, 2001 (RU) 2001119207

(51) Int. Cl. A61B 5/00; A61F 13/15; A61F 13/20

(52) U.S. Cl. 600/362; 604/358; 436/510

(57) ABSTRACT

The invention relates to the medicine and can be used for the prophylaxis and diagnostics of a number of disorders in human organism and also for the revealing of some states accompanied by the changes in the content of diagnostically important metabolites in biological liquids. The object of the invention is to make a basically new sanitary means providing an additional function—testing for the purpose of an organism state analysis—along with his designated purpose and intended for daily use for the patients. The widely used sanitary hygienic article such as pampers or feminine hygienic napkin is provided with the special functional diagnostic means. The functional diagnostic means according to the invention comprises at least one chemical reagent disposed so that it can react with biological liquids secreted by an organism with the following comparison of the change in the content of reference (diagnostically significant) metabolites in liquid biological samples. The number of insulated chemical reagents disposed in random order in said means is selected in dependence of the number of the characteristic for the given disease parameters being determined. The functional diagnostic means is isolated from the adjacent layers and is made so that a biological liquid secreted by an organism will not additionally enter it and a sample of a biological liquid entering the means will not escape outwards. Some variants of the means are suggested.

### FIELD OF THE INVENTION

The invention relates to the medicine and can be used for the prophylaxis and diagnostics of a number of disorders in human organism and also for the revealing of some states accompanied by the changes in the content of diagnostically important metabolites in biological liquids. Among these disorders are diabetes mellitus, ketonuria, L-uria, microalbuminuria, proteinuria, the disorders of protein, fat and carbo-hydrate metabolism and such states as pregnancy.

### BACKGROUND OF THE INVENTION

In spite of the presence of a wide range of diagnostic means and methods until the present time the methods and means of early detection of the unfavourable changes in the organism are very essential. Besides, under certain diseases it is desirable to carry out the continuous monitoring of the content of the reference (signal) metabolites in biological liquid without the constant service of a physician. It is especially important for such patients as infants or the patients having chronic diseases and hard to move (lying patients).

The difficulties of taking necessary samples of biological liquid and solid secretions in such cases are well known.

It is known a wide range of diagnostic means intended for the detection of diagnostically important substances in blood, urine, etc. for which under laboratory conditions it is possible to make rapid analysis on 10 parameters, namely: specific density, leukocytes, acidity (pH), nitrites, protein, erythrocyte, glucose, bilirubin, ketone corpus, urobilinogen, and also allowing to measure the level of AST, cholesterin, amylase, urea, creatine kinase, pancreatic amylase, uric acid, triglycerids, alkaline phosphatase, creatinine, ALT, glucose, bilirubin, hemoglobin etc.

High capacity, the rate of analysis and its simplicity have allowed them to hit the market.

At the same time, as it was mentioned before, these developments can be used not at all times—sometimes their use presents difficulties. Often it is difficult and sometimes it is impossible to take a biological sample and to deliver it for the analysis in time. Besides, the execution of such analysis requires the laboratory with the suitable equipment and high-qualified medical staff.

From the prior art there are known a device and a method of an organism state analysis describing one of the solutions of the problem mentioned above. The known method provides for the determination of chemical compounds in biological liquid secreted by an organism by addition of the falsified agent into a testing strip (PCT WO 92/03731).

The disadvantages of the known device are the following: a complex multi-stage preparation to operate, unprotection of the testing trip from the external conditions and their effect on the patient's organism, the difficulties of its removal for reading the results. Besides, the samples (of the biological liquid secreted by an organism) specially taken for this analysis have to be stored in a specific place protected from light. The time for reading the results appeared on testing strip is very limited and the moment of urination is very difficult to determine definitevely; all the above has a negative effect upon the accuracy of the readings connected with the duration of the exposure of the testing strip to the biologically active liquid. Besides, it is impossible to make an analysis on the set of parameters necessary for the study of the patient's state.

The known device is not a sanitary article and is not intended for urine or blood collection in daily use, for example, for infants or very old people.

There are known napkins which are sanitary articles and can be used for the analysis of an organism state (see H.Γ.3epHOB “ [symbol] [symbol] [symbol] [symbol] ”, 1969, p. 76-77). But taking samples, for example, for phenylketonuria with chloric iron is a complex multi-stage process comprising a preliminary collection of urine, which is rather difficult in case of infants or new-born children, and also additional chemical treatment. When using such napkins urine density is not taken into account, which causes the low accuracy of analysis and the necessity of its repetition. Besides, such analysises require high-qualified staff and are not acceptable in home conditions.

From the prior art it is known a device which can be used with any sanitaric article (see U.S. Pat. No. 4,738,674, 1988). This device comprises an outer layer from non-transparent water-proof material, an inner layer from absorbing material which is made as a combination of a strip from the material of the capillary action type and a substance providing visual indication of wetness when it contacts with this substance. Said substance is contained in the part of the strip. The strip is attached to the inner surface of an outer layer of clothes. Moisture moving along its longitudinal axis due to the capillar effect and reaching the above-mentioned substance provides a visual indication of wetness.

But this device is intended only for the indication of wetness and is not intended for the diagnostics and for an organism state analysis since the construction of the used diagnostical means doesn't allow to carry out this function.

It is known also a sanitary napkin comprising a fluid impervious upper baffle, liquid-proof lower layer, absorbing core and an indicating means adjacent to one of these layers (see U.S. Pat. No. 4,357,939, 1982). The indicating means direct the flow of biological liquid across the width of the napkin and function as an indicator of wetness.

But these napkins can not be used for an organism state analysis since the construction of the diagnostic means being used doesn't allow to carry out this function.

All the above-mentioned sanitary articles don't suggest or provide for the execution of two functions simultaneously: to be a sanitary means intended for daily use and to be a diagnostic means for an organism state analysis and revealing of an early stage of the disease.

### SUMMARY OF THE INVENTION

An object of the present invention is to make a basically new sanitary means providing an additional function —testing for the purpose of an organism state analysis—along with his designated purpose and intended for daily use for the patients including infants in order to reveal the disease at the early stage. The early revealing of, for example, such hard disease as diabetes mellitus is essential in medicine. It especially concerns the families having hereditary predisposition and also for the persons living in the areas with the rapid increase in high carbohydrate food-stuffs consumption and in remote areas without suitable laboratories.

Another object of the present invention is to make simple constructions of such widespread sanitary means as pampers and hygienic napkins which can be used in home conditions for a wide circle. It can be not only the infants but elderly people, lying patients, women of genital period etc.

It was suggested that the disadvantages of known developments can be overcome by the exclusion of difficult stage of sampling and delivering it to the medical laboratory for the analysis. It is suggested to combine the stage of contact of the biological material with the functional diagnostic means when it secrets from the patient's organism. Taking into account the known toxic properties of chemical reagents used in known methods of analysis which doesn't allow to carry out the prolonged contact with skin or mucous membranes, the simple combination with commonly used sanitary-hygienic means is deficient.

It is an object of the present invention to provide such sanitary-hygienic means which can solve all the above-mentioned problems and to eliminate all the demerits.

According to this invention it is provided a sanitary-hygienic article for an organism state analysis in the form of the pampers comprising a front belt section, a back belt section, an extending in longitudinal direction intermediate section including a liquid-permeable upper face layer, an impervious to liquid lower layer, an absorbing core and a functional diagnostic means disposed between the absorbing core and the lower layer, said means comprises at least one chemical reagent disposed so that it can react with the biological liquids secreted by an organism with the following comparison of the change in the content of reference (diagnostically important) metabolites in liquid biological samples, the number of insulated chemical reagents disposed in random order in said means is selected in dependence of the number of characteristic for the given disease parameters being determined, said functional diagnostic means is isolated from the adjacent layers and is made so that a biological liquid secreted by an organism will not additionally enter it and a sample of a biological liquid entering the means will not escape outwards.

According to this invention the same simple construction of sanitary-hygienic article for an organism state analysis is provided in the form of a hygienic napkin.

Said hygienic napkin comprises an extending in longitudinal direction liquid-permeable upper face layer, an impervious to liquid lower layer, an absorbing core and a functional diagnostic means disposed between the absorbing core and the lower layer, said means comprises at least one chemical reagent disposed so that it can react with the biological liquids secreted by an organism with the following comparison of the change in the content of reference (diagnostically important) metabolites in liquid biological samples, the number of insulated chemical reagents disposed in random order in said means is selected in dependence of the number of the characteristic for the given disease parameters being determined, said functional diagnostic means is isolated from the adjacent layers and is made so that a biological liquid secreted by an organism will not additionally enter it and a sample of a biological liquid entering the means will not escape outwards.

For both inventions said functional diagnostic means is provided with at least one additional absorbing element extending beyond it in cross direction and being able to deliver the sample of biological liquid secreted by an organism. Said absorbing element is made from water absorbing material of the capillary action type.

The number of absorbing elements complies with the number of chemical reagents disposed in the functional diagnostic means, each absorbing element is constructionally combined with the corresponding chemical reagent for the delivery of samples of biological liquid secreted by an organism. Besides, the absorbing elements are made in the form of conducting strips or tabs. The distal ends of mentioned strips or tabs are disposed so that they can penetrate into at least one layer disposed above the functional diagnostic means.

The distal ends of the strips or tabs can run along all the layers of the structure or can extend above the absorbing layer (core). Said ends are made so that it is possible to change a slope or an angle of tabs's divergence with reference to longitudinal direction of the layers.

The functional diagnostic means can be made in the form of at least one layer, extending in longitudinal direction and adjacent by its longitudinal tight side to the impervious to liquid lower layer of the structure. Said layer can be divided into sections. Said diagnostic means should include at least one indicating testing strip comprising a detecting and a visualizing agents. Said means can include the set of insulated indicating testing strips each comprising a detecting and a visualizing agents. Each indicating testing strip is disposed in an individual pocket, each section is a pocket with an indicating testing strip disposed in it.

The pocket with the testing strip can be made in the form of a soldered transparent capsule comprising the holes, notches or slots for the disposition of absorbing elemets. The capsule can be not all transparent, it can have transparent windows or at least one transparent window matching with the disposition of corresponding reagent or reagents. Said transparent window or windows intended for visual inspection are disposed on the capsule's side facing the lower impervious layer of the structure, the holes (notches or slots) for absorbing elements are disposed on opposite longitudinal side of said capsule. The hole or holes for the disposition of absorbing elements is made conical.

The pocket can be made with a liquid-tight backstitch along the perimeter on three sides and with one open (without the backstitch) facing the rear side intended for independent permeation of the liquid medium of secretions into the pocket wherein the indicating testing strip is disposed. A visual estimation of the results is carried out by the comparison of obtained color of test-reagent with an indicator scale disposed on the packing. The facing out (an outer from the surface of the body) side of the pocket, i.e. adjacent to the lower layer of the structure, is made from a transparent material, the side of the pocket facing the rear by the side intended for the independent penetration of the biological liquid into the pocket prevents the escape of the biological liquid from the pocket which provides the impossibility of its contact with a body. In this case there are the holes or the notches (slots) on this side of the pocket for the disposition of absorbing elements. In case of the holes the last have conical form for the entry of the absorbing element.

In the specific case when the facing out (an outer from the surface of the body) side of the pocket is made from transparent material, the lower impervious to water layer is also made transparent. In other case said layer can comprise at least one transparent window disposed so that it can be matched with the transparent side of the pocket or transparent window of the capsule. Said transparent window made in the lower layer can be protected from light by the protective opaque to light film which can be removed easily before the use.

Said functional diagnostic means can be made demountable for the following analysis or use. The absorbing elements can be simply detached if they haven't been dissolved previously.

Described variants of the invention can be used with any known sanitary hygienic article, for example, a napkin, an insert into trousers or hospital clothes, and they are not limited by the described pampers or hygienic napkins. Variations and modifications can be effected by a skilled person within the scope of the claimed invention.

### BRIEF DESCRIPTION OF THE DRAWINGS

FIG. 1 is a general view of an absorbing sanitary-hygienic article—the pampers.

FIG. 2 is a general view of an absorbing sanitary-hygienic article—the hygienic napkin.

FIG. 3 is a functional diagnostic means, a top view.

FIG. 4 is a view A-A.

FIG. 5 is a schematic view of the pampers for the determination of one diagnostically important metabolite, for example, glucose.

FIG. 6 is a schematic view of the hygienic napkin for the determination of one diagnostically important metabolite, for example, carrying out the pregnancy test.

FIG. 7 shows the disposition of the absorbing elements, the ends of which enter the notches made in the absorbing core.

FIG. 8 shows the disposition of the absorbing elements, the ends of which pass through the slots made in the absorbing core and are disposed above it.

FIG. 9 is a schematic view of the hygienic pampers with a transparent window in the lower layer.

FIG. 10 is a schematic view of the hygienic napkin with a transparent window in the lower layer.

FIG. 11 is a schematic view of the pampers with a protective film opaque to light.

FIG. 12 is a schematic view of the hygienic napkin with a protective film opaque to light.

### DETAILED DESCRIPTION OF THE PREFERRED EMBODIMENT

The pampers according to the invention comprises 1—a front belt section, 2—a back belt section, 3—an intermediate section, 4—an upper layer permeable to water, 5—an absorbing core, 6—a functional diagnostic means, 7—a lower impervious layer, 8—a testing strip, 9—a chemical reagent, 9a—an imaginary line of the disposition of said chemical reagents, if there is more than 1 reagent, 10—the absorbing elements, 11—a functional diagnostic means made in the form of the layer, 12—a slot (a notch) for the disposition of an absorbing element in the absorbing core, 13—a pocket for the disposition of the testing strip, 14—the backstitch of the pocket, 15—a conical entry on the side of the pocket through which a sample of a biological liquid enters the pocket, 16—an individual section, 17—a protective opaque to light film, 18—the absorbing strips, 19—the absorbing tabs, 20—a transparent window in the lower layer.

The hygienic napkin (see FIGS. 2, 6, 10) according to the invention comprises an upper water-permeable layer 4, an absorbing core 5, a functional diagnostic means 6 and a lower impervious layer 7. The functional diagnostic means comprises a testing strip 8 with a chemical reagent 9, where 9a—an imaginary line of the disposition of said chemical reagents, if there is more than 1 reagent, 10—the absorbing elements, 11—a functional diagnostic means made as a layer, 12—a slot (a notch) for the disposition of an absorbing element in the absorbing core, 13—a pocket for the disposition of the testing strip, 15—the backstitch of the pocket, 15—a conical entry on the side of the pocket through which a sample of a biological liquid enters the pocket, 16—an individual section, 17—a protective opaque to light film, 18—the absorbing strips, 19—the absorbing tabs, 20—a transparent window in the lower layer.

The analysis of an organism state by the suggested construction of sanitary hygienic absorbing means such as the pampers and hygienic napkins has the following steps.

A biological liquid secreted by a person passes through the layers 4 and 6. The liquid-permeable upper layer can have a plurality of holes (not shown). These holes can be disposed uniformly or at random along the length of the layer. The holes are intended for the facilitating of the movement of the liquid secreted by an organism to the upper layer for its permeation into the absorbing core.

Further the liquid comes to a distal end of the absorbing element which extends above the functional diagnostic means or is inside it. The strip or the tabs from water absorbing material of the capillary action type deliver the sample of the biological liquid to the indicating testing strip with the reagent. Due to the presence of a detecting and a visualizing agents in each reagent the visual inspection is possible. The visual estimation of the results is carried out by the comparison of the obtained color (or its change) of the reagent with an indicator scale disposed on the packing. Said scale can be disposed as on the package of the whole pack of the pampers so on the package of the individual pampers in any convenient place.

The functional diagnostic means is a layer extending in longitudinal direction and it can be the size of the layers 3 and 5 or less. Said layer is divided into the sections. Each section being the pocket with the testing strip is insulated from the adjacent section. Each pocket with the testing strip made as a soldered transparent capsule has a hole, which can be conical for the disposition of an absorbing element. The absorbing elements are attached by the ends to the diagnostic means. The other ends passes through the patches or slots made in the layers disposed above the functional diagnostic means. Said notches or slots for said ends of the absorbing elements in dependence of where said ends are terminated can be made in the absorbing core or in the upper water-permeable layer.

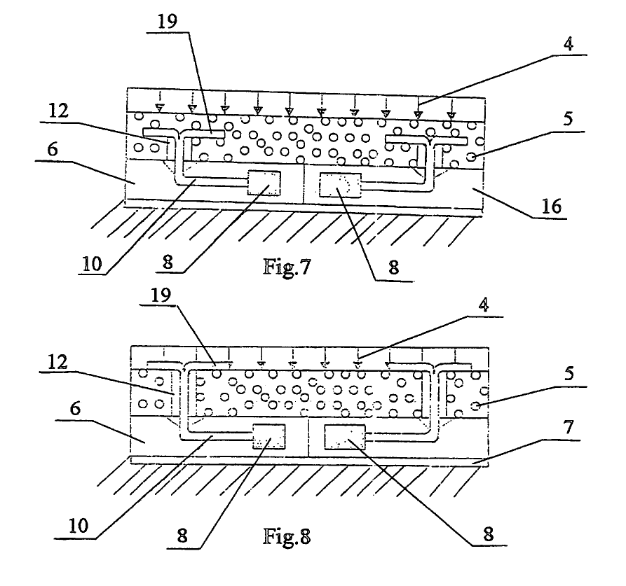

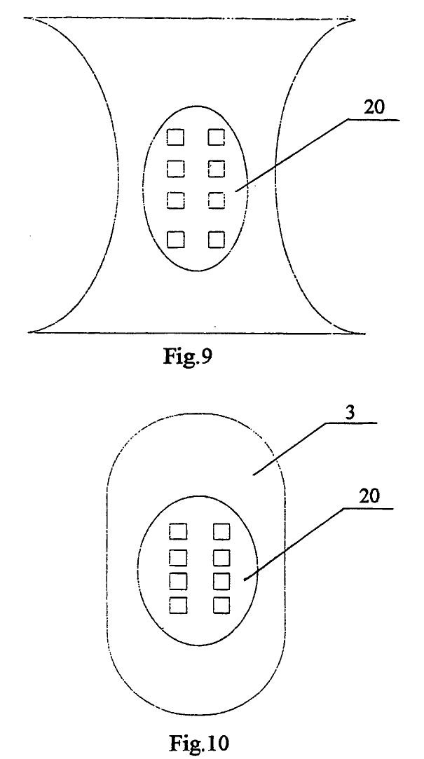

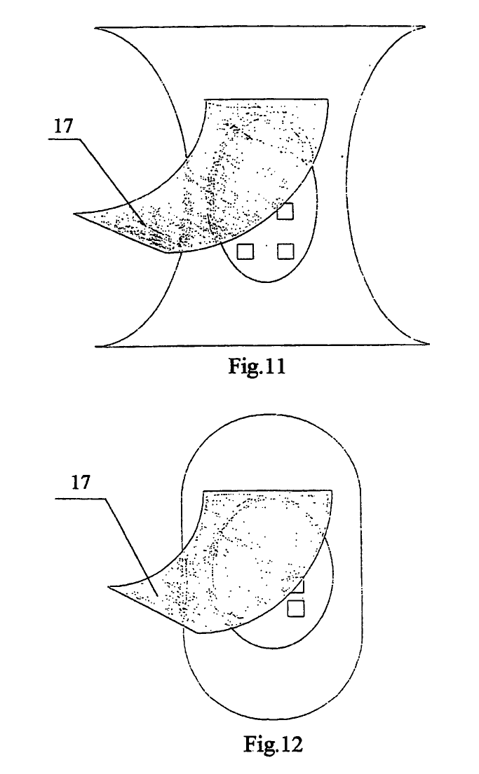

Thus on FIG. 7 it is shown the disposition of the absorbing elements the ends of which enter the layer 5 through the notches made in the absorbing core. On FIG. 8 it is shown the disposition of the absorbing elements the ends of which passes through the notches made in the absorbing core and extend above it.

Urine or other biological liquid due to the capillary action passes through the absorbing element and makes an effect of sealing since due to the equality of the pressures the liquid which has reached the testing strip and has reacted with the test-reagent will be unable to go out. Due to the capillary action the permeated liquid will not allow the additional permeation of the liquid secreted by an organism. The conducting elements made from the material like a blotting-paper after the absorbing of certain amount of liquid swell and seal the hole through which they pass. Besides, it is possible to make the absorbing elements from the material providing gel-forming in a closed space and preventing the exit of the liquid from this closed space.

There are some variants of the disposition of the indicating testing strip with the chemical reagent in the layer of the functional diagnostic means. The functional diagnostic means according to the invention is divided into individual sections, each of which is made in the form of a pocket for the disposition of the testing strip.

Each indicating testing strip is disposed in an individual pocket. Each testing strip is constructively combined with the absorbing element due to the specific construction of the pocket.

If said pocket with the testing strip is made in the form of the soldered capsule the absorbing elements are disposed each in an individual notch or in a special conical hole in the capsule.

Said pocket can be made with a liquid-tight backstitch along the perimeter on three sides and with one open (without the backstitch) facing a body side intended for the independent permeation of the liquid sample into the pocket. The term “pocket” includes such construction which doesn't need special backstitch. The pocket can be made by any way which allows to locate the area of the testing strip in a sanitary-hygienic means and to provide its functions mentioned above. In case of the described construction of the pocket the entry into it is made on the side of the permeation of the liquid sample. Said entry (mouth) of the pocket is made in this case in the form of the conical hole or slot (notch) for the disposition of an absorbing element.

So due to the original construction of the pocket and to the material of an absorbing element the entry into the pocket is closed for the additional permeation of the biological liquid just as into the pocket so outward the pocket.

Each pocket with the testing strip, as it was mentioned before, can be made in the form of a soldered capsule with the holes or slots for the absorbing elements. The conical form of said hole for the disposition of an absorbing element in this case is optimum since it increases the effect of localization (the effect of non-spilling) of the liquid.

Said capsule is made transparent or is provided with at least one transparent window made on the side of the lower layer. Through this window a visual estimation of change in reagent's color and following monitoring of this change with the help of the scale are possible. The scale can be disposed as on the package of the whole pack of the pampers so on the package of the individual pampers. Test scale should conform to given batch of chemical reagents.

If necessary, the diagnostic capsule, i.e. the capsule with the diagnostic means, can be withdrawn and tested in suitable apparatus which gives more precise data in comparison with the visual comparison with test-scale.

Said diagnostic capsule can be joined with the lower layer or with the absorbing layer by an adhesive, thermo-melting means or a clue effective on short-term pressing. The methods of joining are well known for a skilled person.

If the capsule and the lower water-impervious layer are made from transparent material or have transparent windows, aligned relative to each other, the visual inspection can be made not taking off the pampers thus facilitating the stated problem.

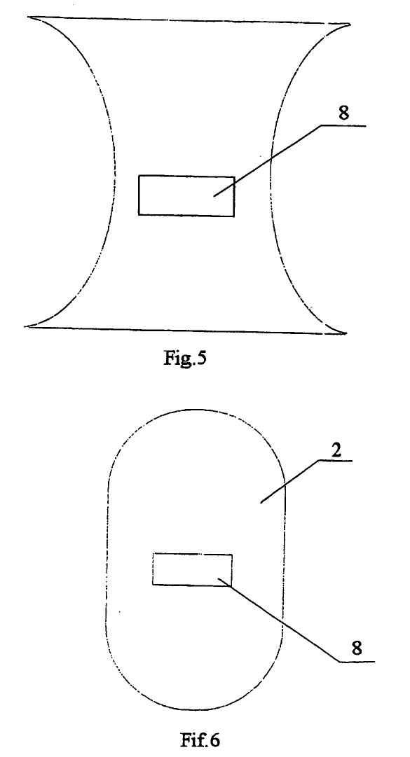

The pampers can be made for the determination of one diagnostically important metabolite (for example, glucose). The disposition of the testing strip in this case is shown on FIG. 5.

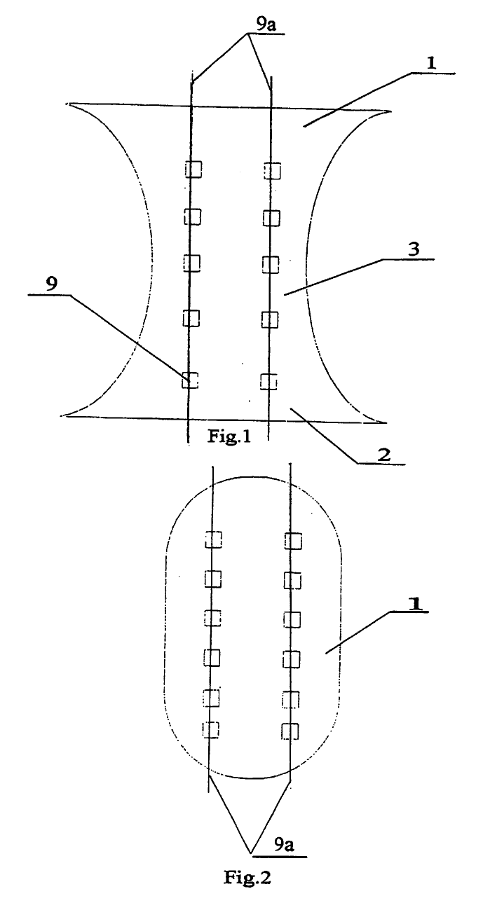

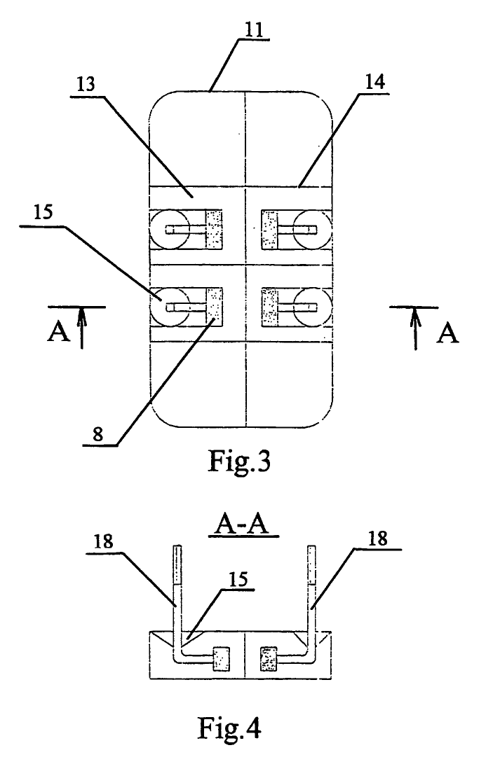

The other variant of the pampers intended for the plural analysis is shown on FIG. 1. For example, the testing strip is disposed in two lines with 5 test-determinations in each line.

It is possible to determine simultaneously:

```
  1-the density
  2-pH (acidity)
  3-z (leukocytes)
  4-nitrates
  5-protein
  6-glucose
  7-ketones
  8-urobilinogen
  9-bilirubin
10-erythrocytes
```

On FIG. 6 it is shown a feminine hygienic napkin for daily use provided with the test on pregnancy.

The presence or absence of the colored stripe on the testing strip after the ingress of urine indicates the presence or absence of pregnancy.

On FIG. 2 it is shown a feminine hygienic napkin for daily use provided with the set of tests (see above).

So with the help of described sanitary-hygienic absorbing articles it is possible to carry out an analysis of patient's state accompanied by the change in the content of reference (diagnostically important) metabolites in liquid biological samples. Said method includes the contact of biological sample with the functional diagnostic means having at least one indicating testing strip comprising an indicating and a visualizing agents and subsequent comparison of the color of at least one testing strip with standard scale disposed on the package of the sanitary-hygienic article for the analysis of the organism state, the contact is carried out with the functional diagnostic means previously disposed in one of the described absorbing sanitary-hygienic article.

Said method allows to analyze patient's state selected from the group including such disorders as the disorders of protein, fat and carbo-hydrate metabolism, pregnancy, first exhibitions of diabetes mellitus etc.

Further the invention will be illustrated by the examples intended exclusively for the confirmation of the possibility of application of the invention. It will be understood that the present invention is not limited to the described embodiments and various methods and means can be used within the spirit and scope of the invention defined in the claims.

The invention doesn't seek to exclude the physicians. Moreover, the obligatory teaching by the physicians is supposed how to use the suggested sanitary-hygienic means. The obtained results must be estimated as the instructions to be examined by the physician.

### EXAMPLE 1.

The patient A, 37 years. She has an insulin dependent diabetes mellitus during 11 years. The disease has an unstable character which makes difficulties in the selection of adequate insulinization. The intake of exogenous insulin varies from 58 units per day to 30 units per day. In the last two years there have been found the pathologic changes in kidneys defined as nephropathy. A proteinuria upper level factor of 10-12 was found in urine analysis. The continuous monitoring of glucose level in blood is necessary. The physician had suggested to use a feminine hygienic napkin provided with the described functional diagnostic means with the testing strip for the determination of glucose in urine. The number of requests to the physician has decreased. All the requests were opportune and stipulated. The patient has no grievances against the quality of napkins, she is satisfied with them since they allow her to save her time, to estimate her own state more adequate and to control the amount of insulin.

More than 100 patients having diabetes mellitus have carried out the independent monitoring. The obtained positive effect corresponds to that of patient A.

### EXAMPLE 2.

The parents had carried out the continuous monitoring of a first year child with the help of the pampers provided with the described functional diagnostic means with a test of urine analysis on 10 positions. On the 6^{th} month of his life the child had fallen ill with an acute respiratory disease complicated by the infection of urinary tracts. This complication is diagnosed in time which allows to provide an opportune correction of the course of medical treatment and more rapid recovery.

More than 500 families had carried out the independent monitoring with the use of pampers provided with the described functional diagnostic means with testing strips which allows to reveal the early exhibition of diseases for 4 infants.

### EXAMPLE 3.

The parents had carried out the continues monitoring of a first year child with the help of pampers provided with the test on glucose.

The hereditary diabetes mellitus is the case. Due to the monitoring the parents had noticed the first exhibitions of the disease on the 8^{th} month of their child's life which allows to hospitalize him into the department of endocrinology, where the pediatric examination had been carried out and the treatment had been prescribed.

The parents of more than 50 children had carried out the monitoring, diabetes mellitus was found once.

### EXAMPLE 4.

A woman, 22 years, has 1 child (10 months old). Every day she had used hygienic, napkins provided with the pregnancy testing strip. Having found the positive pregnancy test she had applied to the gynecologist. Taking into account the early periods of pregnancy the abortion had been carried out in spared conditions and it didn't have an effect on her health.

The napkins with the test were used by 150 women. The effect obtained corresponds to that of this patient.

### EXAMPLE 5.

In standard pampers the applicable section of the layer of the functional diagnostic means is made in the form of a pocket measuring 2×10 cm in the area adjacent to urethra by stitching on three sides. The pocket side, equal 2 cm, the nearest to urethra, is left opened for the permeation of urine into the pocket by an absorbing element connected with said testing strip. The pocket is filled with a wet absorbing standard for pampers material. The pocket has a transparent side matched with a transparent lower moisture impervious layer through which a visual monitoring is carried out.

The sanitary hygienic articles combined with the functional diagnostic means comprising at least one chemical reagent disposed on the indicating testing strip disposed in the construction of the means help to monitor the state of health of the first year children, seriously ill patients in critical state, after insult patients which need a continuous help and monitoring. Said means are useful for the determination of the beginning of many disease including the diseases of kidneys, liver, gastroenteric tract, pancreas, metabolism, diabetes mellitus etc.

The advantages of the invention are its simplicity, the possibility of testing in home conditions, the saving of patient's and physician's time, qualitative monitoring minimizing the subjective factor (the patient's complaints, not confirmed by the objective features of a disease), the revealing of the beginning of a disease before the first clinical exhibition.

1. A sanitary hygienic article for an organism state analysis made in the form of pampers comprising a front belt section, a back belt section, an extending in longitudinal direction intermediate section including a liquid-permeable upper face layer, an impervious to liquid lower layer, an absorbing core and a functional diagnostic means disposed between the absorbing core and the lower layer, said means comprises at least one chemical reagent disposed so that it can react with the biological liquids secreted by an organism with the following comparison of the change in the content of reference (diagnostically important) metabolites in liquid biological samples, the number of insulated chemical reagents disposed in random order in said means is selected in dependence of the number of characteristic for the given disease parameters being determined, said functional diagnostic means is isolated from the adjacent layers and is made so that a biological liquid secreted by an organism will not additionally enter it and a sample of a biological liquid entering the means will not escape outwards.

2. A sanitary hygienic article according to claim 1 characterized in that the functional diagnostic means is provided with at least one additional absorbing element extending beyond it in cross direction and being able to deliver the sample of biological liquid secreted by an organism.

3. A sanitary hygienic article according to claim 2 characterized in that said absorbing element is made from water absorbing material of the capillary action type.

4. A sanitary hygienic article according to claim 3 characterized in that the number of absorbing elements complies with the number of chemical reagents disposed in the functional diagnostic means.

5. A sanitary hygienic article according to claim 4 characterized in that each absorbing element is constructionally combined with the corresponding chemical reagent.

6. A sanitary hygienic article according to claim 5 characterized in that the absorbing elements are made in the form of conducting strips or tabs.

7. A sanitary hygienic article according to claim 6 characterized in that the distal ends of mentioned strips or tabs are disposed so that they can penetrate into at least one layer disposed above the functional diagnostic means.

8. A sanitary hygienic article according to claim 6 characterized in that the distal ends of the strips or tabs run along all the absorbing core and extend above it.

9. A sanitary hygienic article according to claim 7 or 8 characterized in that the distal ends of the strips or tabs are made so that it is possible to change a slope or an angle of tabs's divergence with reference to longitudinal direction of the layers.

10. A sanitary hygienic article according to any one of claims 1-9 characterized in that the functional diagnostic means includes at least one indicating testing strip comprising a detecting and a visualizing agents.

11. A sanitary hygienic article according to claim 10 characterized in that the functional diagnostic means comprises a set of insulated indicating testing strips each comprising a detecting and a visualizing agents.

12. A sanitary hygienic article according to any one of the claims 1-11 characterized in that the functional diagnostic means is made in the form of at least one layer, extending in longitudinal direction and adjacent by its longitudinal tight side to the impervious to liquid lower layer of the structure.

13. A sanitary hygienic article according to claim 12 characterized in that said layer comprises at least one section made in the form of a pocket for the disposition of an indicating testing strip.

14. A sanitary hygienic article according to claim 13 characterized in that each indicating testing strip is disposed in an individual pocket.

15. A sanitary hygienic article according to claim 14 characterized in that the pocket is made with a liquid-tight backstitch along the perimeter on three sides and with one open (without the backstitch) facing the rear side intended for independent penetration of the liquid medium of secretions into the pocket wherein the indicating testing strip is disposed.

16. A sanitary hygienic article according to claim 15 characterized in that the pocket's side adjacent to the lower moisture impervious layer is made from transparent material or has at least one transparent window matching with the disposition of corresponding reagent.

17. A sanitary hygienic article according to any one of the claims 13-16 characterized in that the side of the pocket facing the rear by the side intended for the independent permeation of the biological liquid into the pocket prevents the escape of the biological liquid from the pocket which provides the impossibility of its contact with a body.

18. A sanitary hygienic article according to claim 17 characterized by that the side of the pocket intended for independent permeation of the biological liquid into the pocket has a conical entry for the disposition of the absorbing elements.

19. A sanitary hygienic article according to claim 14 characterized in that each pocket with the testing strip is made in the form of a soldered capsule.

20. A sanitary hygienic article according to claim 19 characterized in that the capsule is made transparent or has at least one transparent window matched with the position of corresponding reagent.

21. A sanitary hygienic article according to claim 20 characterized in that the capsule has the holes for the disposition of the absorbing elements.

22. A sanitary hygienic article according to claim 21 characterized in that the holes for the disposition of the absorbing elements are made conical.

23. A sanitary hygienic article according to any one of the claims 1-22 characterized in that the liquid impervious lower layer is made transparent.

24. A sanitary hygienic article according to any one of the claims 1-22 characterized in that the liquid impervious lower layer has at least one transparent window matched with the position of corresponding window in the capsule or in the side of the pocket for the visual reading of the results.

25. A sanitary hygienic article according to any one of the claims 1-24 characterized in that the functional diagnostic means is made demountable for the following analysis.

26. A sanitary hygienic article for an orghanism state analysis made in the form of a napkin comprising an extending in longitudinal direction liquid-permeable upper face layer, an impervious to liquid lower layer, an absorbing core and a functional diagnostic means disposed between the absorbing core and the lower layer, said means comprises at least one chemical reagent disposed so that it can react with the biological liquids secreted by an organism with the following comparison of the change in the content of reference (diagnostically important) metabolites in liquid biological samples, the number of insulated chemical reagents disposed in random order in said means is selected in dependence of the number of the characteristic for the given disease parameters being determined, said functional diagnostic means is isolated from the adjacent layers and is made so that a biological liquid secreted by an organism will not additionally enter it and a sample of a biological liquid entering the means will not escape outwards.

27. A sanitary hygienic article according to claim 26 characterized in that the functional diagnostic means is provided with at least one absorbing element extending beyond it in cross direction and being able to deliver the sample of biological liquid secreted by an organism.

28. A sanitary hygienic article according to claim 27 characterized in that said absorbing element is made from water absorbing material of the capillary action type.

29. A sanitary hygienic article according to claim 28 characterized in that the number of absorbing elements complies with the number of chemical reagents disposed in the functional diagnostic means.

30. A sanitary hygienic article according to claim 29 characterized in that each absorbing element is constructionally combined with the corresponding chemical reagent.

31. A sanitary hygienic article according to claim 30 characterized in that the absorbing elements are made in the form of conducting strips or tabs.

32. A sanitary hygienic article according to claim 31 characterized in that the distal ends of mentioned strips or tabs are disposed so that they can penetrate into at least one layer disposed above the functional diagnostic means.

33. A sanitary hygienic article according to claim 32 characterized in that the distal ends of the strips or tabs run along all the absorbing core and extend above it.

34. A sanitary hygienic article according to claim 32 or 33 characterized in that the distal ends of the strips or tabs are made so that it is possible to change a slope or an angle of tabs's divergence with reference to longitudinal direction of the layers.

35. A sanitary hygienic article according to any one of claims 27-34 characterized in that the functional diagnostic means includes at least one indicating testing strip comprising a detecting and a visualizing agents.

36. A sanitary hygienic article according to claim 35 characterized in that the functional diagnostic means comprises a set of insulated indicating testing strips each comprising a detecting and a visualizing agents.

37. A sanitary hygienic article according to any one of the claims 26-36 characterized in that the functional diagnostic means is made in the form of at least one layer, extending in longitudinal direction and adjacent by its longitudinal tight side to the impervious to liquid lower layer of the structure.

38. A sanitary hygienic article according to claim 37 characterized in that said layer comprises at least one section made in the form of a pocket for the disposition of an indicating testing strip.

39. A sanitary hygienic article according to claim 38 characterized in that each indicating testing strip is disposed in an individual pocket.

40. A sanitary hygienic article according to claim 39 characterized in that the pocket is made with a liquid-tight backstitch along the perimeter on three sides and with one open (without the backstitch) facing the rear side intended for independent permeation of the liquid medium of secretions into the pocket wherein the indicating testing strip is disposed.

41. A sanitary hygienic article according to claim 40 characterized in that the pocket's side adjacent to the lower layer is made from transparent material or has at least one transparent window matching with the disposition of corresponding reagent.

42. A sanitary hygienic article according to claim 40 or 41 characterized in that the side of the pocket facing the rear by the side intended for the independent permeation of the biological liquid into the pocket prevents the escape of the biological liquid from the pocket which provides the impossibility of its contact with a body.

43. A sanitary hygienic article according to claim 42 characterized by that the side of the pocket intended for independent permeation of the biological liquid into the pocket has a conical entry for the disposition of the absorbing elements.

44. A sanitary hygienic article according to claim 39 characterized in that each pocket with the testing strip is made in the form of a soldered capsule.

45. A sanitary hygienic article according to claim 44 characterized in that the capsule is made transparent or has at least one transparent window matched with the position of corresponding reagent.

46. A sanitary hygienic article according to claim 45 characterized in that the capsule has the holes for the disposition of the absorbing elements.

47. A sanitary hygienic article according to claim 46 characterized in that the holes for the disposition of the absorbing elements are made conical.

48. A sanitary hygienic article according to any one of the claims 26-47 characterized in that the liquid impervious lower layer is made transparent.

49. A sanitary hygienic article according to any one of the claims 26-47 characterized in that the liquid impervious lower layer has at least one transparent window matched with the position of corresponding window in the capsule or in the side of the pocket for the visual reading of the results.

50. A sanitary hygienic article according to any one of the claims 26-49 characterized in that the functional diagnostic means is made demountable for the following analysis.

51. A method of the analysis of a patient's state accompanied by the change in the content of diagnostically important metabolites in liquid biological samples comprising the contact over a period of time of biological liquid with the functional diagnostic means having at least one indicating testing strip with a detecting and a visualizing agents and following comparison of said at least one testing strip with standard scale applied to the packing of the sanitary hygienic means for an organism state analysis, the contact is carried out with the functional diagnostic means first disposed in a certain way in the sanitary hygienic article according to claim 1 or 26.
